(19) United States
(12) Patent Application Publication
(10) Pub. No.: US 2026/0165717 A1
(43) Pub. Date: Jun. 18, 2026

(54) CUTTING GUIDE WIRE AND THROMBUS SUCTION SYSTEM

(71) Applicant: Shanghai INT Medical Instruments Co., Ltd., Shanghai (CN)

(72) Inventors: Hongxin ZHOU, Shanghai (CN); Dongke LIANG, Shanghai (CN); Wei HUAN, Shanghai (CN); Chunming SUN, Shanghai (CN); Guang CHENG, Shanghai (CN)

(21) Appl. No.: 18/712,461

(22) PCT Filed: May 30, 2023
(86) PCT No.: PCT/CN2023/097174

§ 371 Date: May 22, 2024

(30) Foreign Application Priority Data
Nov. 17, 2022 (CN) 202211439559.5

(51) Int. Cl. A61B 17/22; A61B 17/3207

(52) U.S. Cl. CPC A61B 17/22; A61B 2017/22038; A61B 2017/22079; A61B 2017/320775

(57) ABSTRACT

The present application provides a cutting guide wire and a thrombus suction system. The cutting guide wire includes a guide wire body and a thrombus cutting member, a distal end of the guide wire body is suitable for being arranged in a suction catheter in a passing-through mode, the thrombus cutting member is connected to the distal end of the guide wire body and suitable for being placed in the suction catheter, and the thrombus cutting member has a cutting state of moving in the suction catheter to cut a thrombus in the suction catheter under a driving action of the guide wire body; wherein the thrombus cutting member includes a cutting portion, and a cross-sectional area of the cutting portion gradually increases in a direction from a distal end thereof to a proximal end thereof. The thrombus cutting member is connected to the distal end of the guide wire body and includes the cutting portion, the cross-sectional area of the cutting portion gradually increases in the direction from the distal end thereof to the proximal end thereof, so when the thrombus cutting member is placed in the suction catheter to move so as to cut the thrombus in the suction catheter through the guide wire body, the cutting portion in a tip shape can cut and crush the thrombus blocking the suction catheter, and an interior of the suction catheter can be prevented from being blocked.

### 2. CLAIMS OF THE UTILITY MODEL

(1) A guide wire, characterized in that a front end of the guide wire is provided with a cutter, and a screw vane arranged along a concentric axis of the guide wire is provided on a rear side of the cutter.
(2) The guide wire according to claim 1, wherein, the screw vane is made of metal or plastic.
(3) The guide wire according to claim 1 or claim 2, wherein, a core wire of the guide wire is made of a shape memory alloy.

### 3. DETAILED DESCRIPTION OF THE UTILITY MODEL

### Application Fields in Industry

The present utility model relates to a guide wire with a screw vane, in particular, relates to a guide wire with a screw vane for cleaning thrombi in blood vessels, atherosclerotic plaques (atheroma) in the interior of the biliary tract and the like, or gallstones and the like in the bile duct, and delivering them to the exterior.

### Background of the Utility Model

Traditionally, for the cleaning of atherosclerotic plaques (atheroma) or gallstones formed in the bile duct, when these substances are embedded in the duct wall, they need to be first cut by a cutter and then are removed from the body by using tools such as stone basket forceps or alligator mouth forceps. For free stones, similarly, after the stones have been broken into pieces, it is necessary to use forceps to remove the pieces of stones.

However, when forceps are used to remove cleared substances obtained by cutting or breaking atherosclerotic plaques or gallstones embedded in the duct wall one by one, it is necessary to repeatedly perform the operation of grasping and removing them one by one. This operation not only requires proficient mastery of the operation skills, but also has the disadvantage of taking a long time to grasp.

Therefore, in order to solve problems or shortcomings, after extensive research, the inventors of the present utility model have developed an apparatus that can remove the above-mentioned cleared substances in a simple and continuous way.

### Objective of the Utility Model

Therefore, the objective of the present utility model is to provide a guide wire that can remove cleared substances such as atherosclerotic plaques or gallstones in a simple and continuous way.

### Means to Solve the Problem

The above-mentioned objective of the present utility model is achieved by the following (1) to (3).
(1) A guide wire, characterized in that a front end of the guide wire is provided with a cutter, and a screw vane arranged along the concentric axis of the guide wire is provided on a rear side of the cutter.
(2) The guide wire according to claim 1, wherein, the screw vane is made of metal or plastic.
(3) The guide wire according to claim 1 or claim 2, wherein a core wire of the guide wire is made of a shape memory alloy.

In the following, the structure of the present utility model is described in detail. A cutter is provided on a front end portion of the guide wire of the present utility model, and a screw vane (also called a screw) for conveying gallstones and other foreign matters that have been cut off by the cutter is provide on a rear side of the cutter. As a cutter, cutters commonly used in the art may be employed without any special restrictions, but it is preferred to use the cutter described in the following embodiments.

In addition, in the guide wire with a screw vane, the screw vane is arranged along a concentric axis of the guide wire. The size of the screw vane may vary, depending on the diameter of the guide wire or the thickness of the applicable blood vessel, but the cross-sectional diameter of the screw vane should be at least 0.4 mm, with the upper limit determined by the thickness of the blood vessel. Preferably 0.6 mm to 2.0 mm.

In addition, the length of the screw vane is within the range of 20 mm to 100 mm, especially within the range of 50 mm to 80 mm, which is more suitable.

Materials for making the screw vane of the present utility model include stainless steel, gold, platinum, rhodium, iridium and other metals or alloys, or plastics. The fixation between the screw vane and the guide wire can be achieved by using weld materials such as silver solder, which are commonly used in the art. On the other hand, when the screw vane is made of plastic, it is preferred to use adhesive to fix the screw vane.

The position of the screw vane of the present utility model is set on the rear side of the cutter, specifically, it is preferred to be set within a range of 10 mm directly behind the connection portion of the cutter.

As a guide wire, guide wires of various known materials or structures can be used. The following guide wires are particularly preferred: guide wires composed solely of metal wires, guide wires using helical springs, guide wires combining helical springs and core wires, guide wires that are made flexible by forming the front end portion of the guide wire with metal wires or helical springs, guide wires with the front end portion made thinner than the rest of the guide wire, and so on.

Materials used to make these guide wires include stainless steel wires, piano wires, super elastic metal wires, shape memory alloy wires, plastics, and the like. Among them, the super elastic metal wire of the shape memory alloy wire may be of a conventional model in the art.

The diameter of the guide wire is determined by the purpose of use, usually ranging from 0.3 mm to 0.7 mm, but is not limited to this.

The guide wire of the present utility model can be partially or fully covered with a coating layer. Coating materials can be selected from polyethylene, polyvinyl chloride, polypropylene, polyamide, polyurethane, fluororesin, silicon resin, etc.

Moreover, it is advisable to add antithrombotic agents such as heparin and urokinase into these coating layers.

In addition, as a coating layer, antithrombotic materials such as hydroxymethacrylate-styrene copolymer can also be used.

In addition, as needed, X-ray contrast agents such as salts of Ba, W, Bi, Pb and other metal (such as sulphates, carbonates, etc.) can also be added.

The guide wire of the present utility model is suitable for the cleaning of atherosclerotic plaques or gallstones in the liver, bile ducts, etc., and can also be used in other parts of the organism with similar functions.

When the guide wire of the present utility model is used, due to the fact that the guide wire can be used as a conveying path for atherosclerotic plaques or stone pieces that have been cut off, it is preferred to be used in combination with a catheter or a sheath tube, and the guide wire can be used by rotating it in the catheter. The rotation mode can be manual or motor-driven, etc. In addition, when using this guide wire, in order to facilitate the removing of the cleared substances, suction can also be carried out simultaneously by intravascular decompression.

### Functions of the Present Utility Model

In the present utility model, the guide wire with a screw vane can be rotated, so that the cutter fixed at its front end portion rotates to cut off the atherosclerotic plaques and the like in the affected areas. The obtained cleared substances are moved backward and expelled under the action of the rotating screw vane.

Therefore, the cleared substances can be moved and expelled in a simple and continuous way.

The present utility model is further described in detail below with reference to the accompanying drawings.

### Embodiments

The present utility model is described in detail in the following with reference to the accompanying drawings, but this is only one implementing way of the present utility model and the present utility model is not limited to this.

FIG. 1 is a cross-sectional view of the guide wire with a screw vane of the present utility model, a cutter 3 is provided at the front end of the guide wire 1, and a screw vane 2 is provided immediately behind the cutter 3. The two are fixed together by brazing materials.

Specifically, at the front end of the guide wire 1 with a diameter of 0.5 mm, a cutter with a diameter of 0.9 mm and a length of 10 mm is fixed on a concentric circle of the guide wire. On a surface of the guide wire, a screw vane with a diameter of 0.8 mm and a length of 120 mm is arranged in a spiral shape along a concentric circle, and both ends of the screw vane are fixed with weld materials. The front end of the guide wire protrudes by a length of 20 mm from a cutting edge portion of the cutter.

In addition, the cutter and the screw vane are fixed with the guide wire as a shaft. In other embodiments, the cutter and the screw vane can act freely and separately.

FIG. 2 is the front view of the guide wire of the present utility model. The guide wire 1 is located at the center of the cutter 3, and a tip end of the cutting edge portion 31 is arranged along a concentric circle of the guide wire 1 to prevent the cutter from wobbling during use.

In FIG. 1, the guide wire of the present utility model is preferably used in conjunction with a sheath tube (cylindrical object) or a catheter shown by the dotted line. It is more preferable that the guide wire of the present utility model is moved with the cutter covered by the sheath tube before reaching the affected area, and the cutter is pushed out from the sheath tube for use when the guide wire reaches the affected area.

When in use, the guide wire is rotated at the affected area, so that the cutter fixed at the front end portion rotates to cut off atherosclerotic plaques and the like at the affected area. The obtained cleared substances are moved backward and expelled by means of the rotating screw vane.

Further, the cutter used in the present utility model has shapes shown in FIG. 3. In FIG. 3(a), the guide wire is covered by two conical cutting edges of the cutter distributed along a concentric circle. Alternatively, in FIG. 3(b), a single conical cutting edge is connected to the front end of the guide wire.

### Effects of the Utility Model

In the present utility model, when the guide wire with the screw vane is rotated, the cutter at the front end thereof rotates to cut off atherosclerotic plaques and the like. The obtained cleared substances are moved backward and expelled by means of the rotating screw vane.

Therefore, the cleared substances can be moved and expelled in a simple and continuous way

### 4. BRIEF DESCRIPTION OF THE ACCOMPANYING DRAWINGS

FIG. 1 shows a cross-sectional view of the guide wire with a screw vane of the present utility model.

FIG. 2 shows a front view of the guide wire with a cutter at its front end, in the guide wire of the present utility model.

FIG. 3(a) and 3(b) shows oblique views of the structure of the cutter.

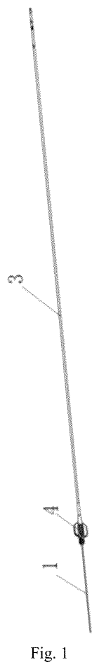

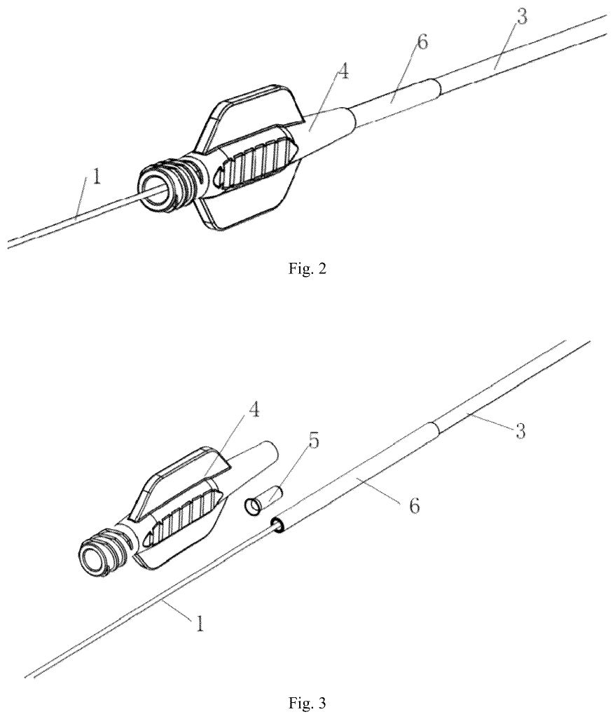

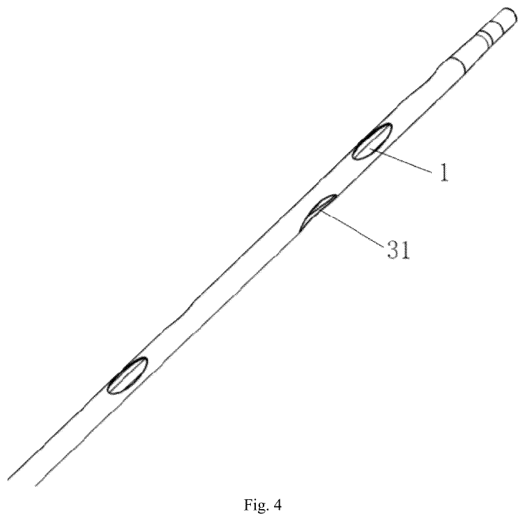

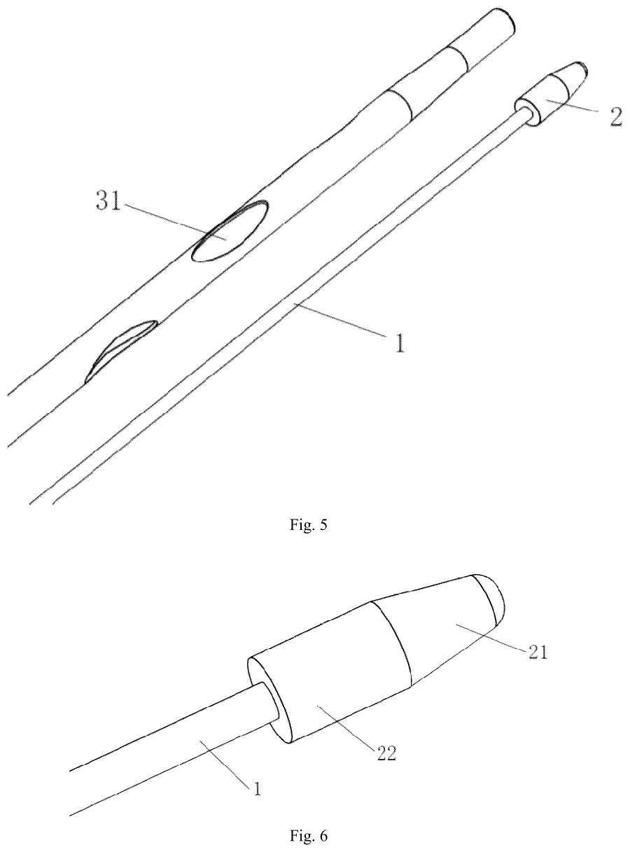

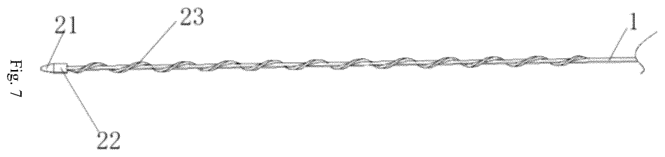

### REFERENCE SYMBOLS

1 guide wire
2 screw vane or screw
3 cutter
31 cutting edge portion
4 weld material
5 sheath tube or cylindrical object

1. A cutting guide wire, comprising:
a guide wire body (1), with a distal end of the guide wire body (1) configured to be arranged in a suction catheter (3) in a passing-through mode; and
a thrombus cutting member (2), connected to the distal end of the guide wire body (1), the thrombus cutting member (2) being configured to be placed in the suction catheter (3), and the thrombus cutting member (2) having a cutting state of moving in the suction catheter (3) to cut a thrombus in the suction catheter (3) under a driving action of the guide wire body (1);
wherein the thrombus cutting member (2) comprises a cutting portion (21), and a cross-sectional area of the cutting portion (21) gradually increases in a direction from a distal end thereof to a proximal end thereof.

2. The cutting guide wire according to claim 1, wherein a lateral wall surface of the cutting portion (21) is a circular truncated cone surface or a paraboloid surface.

3. The cutting guide wire according to claim 1, wherein a plurality of protrusions and/or recesses are arranged on a peripheral side surface of the cutting portion (21).

4. The cutting guide wire according to claim 3, wherein the thrombus cutting member (2) further comprises a connecting portion (22), and the connecting portion (22) is connected between the guide wire body (1) and the cutting portion (21).

5. The cutting guide wire according to claim 4, wherein a cross-sectional area of the connecting portion (22) gradually increases in a direction from a proximal end thereof to a distal end thereof.

6. The cutting guide wire according to claim 5, wherein the connecting portion (22) is of a circular truncated cone structure or a paraboloid structure.

7. The cutting guide wire according to claim 3, wherein the thrombus cutting member (2) further comprises a spiral portion (23), the spiral portion (23) is connected to the connecting portion (22), and the guide wire body (1) is arranged inside the spiral portion (23) in a passing-through mode.

8. The cutting guide wire according to claim 7, further comprising a rotary knob connected to a proximal end of the guide wire body (1) so as to be able to drive the guide wire body (1) to rotate.

9. A thrombus suction system, comprising the cutting guide wire according to claim 1.

10. The thrombus suction system according to claim 9, comprising a negative pressure source, a connector, the cutting guide wire, a suction catheter (3) and a catheter handle (4), the connector being connected between the negative pressure source and the suction catheter (3), the catheter handle (4) being connected to a proximal end of the suction catheter (3), the cutting guide wire configured to be placed in the suction catheter (3), and a plurality of lateral holes (31) are formed in a lateral wall of a distal end of the suction catheter (3).
